(19) United States
(12) Patent Application Publication
(10) Pub. No.: US 2026/0345111 A1
(43) Pub. Date: Nov. 5, 2026

(54) METHOD, APPARATUS, AND SYSTEM FOR PREDICTING A PULMONARY ARTERIAL HYPERTENSION CONDITION OF A SUBJECT

(71) Applicant: The Hong Kong University of Science and Technology, Hong Kong (CN)

(72) Inventors: Xiaomeng LI, Hong Kong (CN); Jiewen YANG, Hong Kong (CN)

(21) Appl. No.: 19/488,913

(22) PCT Filed: Sep. 13, 2024
(86) PCT No.: PCT/CN2024/118695

§ 371 Date: Dec. 1, 2025

Related U.S. Application Data
(60) Provisional application No. 63/582,844, filed on Sep. 14, 2023.

(51) Int. Cl. A61B 8/08; A61B 8/00; G06T 7/62

(52) U.S. Cl. CPC A61B 8/0883; A61B 8/5223; G06T 7/62; G06T 2207/10016; G06T 2207/10132; G06T 2207/30048

(57) ABSTRACT

The present disclosure relates generally to a method, apparatus, and system for predicting a pulmonary arterial hypertension condition of a subject, the method comprising: measuring, by a processor, a morphological feature of a region of a heart of the subject from a plurality of echocardiogram videos, each of the plurality of echocardiogram videos showing the heart at a different viewing angle, the morphological feature relating to the pulmonary arterial hypertension condition; and generating, by the processor, a prediction score representing a likelihood of the subject having the pulmonary arterial hypertension condition from the morphological feature.

### CROSS REFERENCE TO RELATED APPLICATIONS

This application claims priority to, and the benefit of, U.S. Provisional Patent Application No. 63/582,844 filed on Sep. 14, 2023, the disclosure of which is incorporated by reference herein in its entirety.

### TECHNICAL FIELD

The present disclosure relates generally to method, apparatus, and system for predicting a medical condition of a subject, and more particularly a pulmonary arterial hypertension condition of a subject.

### BACKGROUND

Pulmonary arterial hypertension (PAH) is a rare and progressive cardiovascular disorder characterised by high blood pressure in the pulmonary arteries, which can lead to premature death if left untreated. Early detection of PAH is crucial as appropriate treatment in the early stages can significantly improve patients' longevity and quality of life. However, diagnosing PAH effectively is challenging since the disease is often clinically silent in its early stages, resulting in delayed diagnosis and treatment.

Currently, right heart catheterisation (RHC) is considered the gold standard for diagnosing PAH, by providing direct haemodynamic measurements. However, RHC is an invasive procedure with risk of complications such as bruising, excessive bleeding, and potential lung collapse. Moreover, its reliance on highly skilled physicians limits its widespread use for PAH screening initiatives.

In contrast, image-based PAH diagnostic methods using imaging techniques such as echocardiography, chest radiographs, and cardiovascular magnetic resonance imaging offer safer, non-invasive alternatives to RHC. However, these methods rely on the expertise of the interpreting physician, which can lead to variability and potential misdiagnosis. Additionally, chest radiographs and cardiovascular magnetic resonance imaging, despite being non-invasive, present challenges such as radiation exposure and long imaging time required, making them less suitable for routine screening purposes.

Furthermore, existing image-based PAH diagnostic methods primarily focus on distinguishing between PAH and non-PAH cases and are incapable of accurately measuring key parameters such as mean pulmonary artery pressure (mPAP) and assessing different PAH conditions such as severity of PAH. At present, diagnostic methods using echocardiography only rely on single-view echocardiogram videos, which may provide an incomplete representation of cardiac morphology. Consequently, the diagnostic capability of such methods is limited due to potential inaccuracies.

Accordingly, there exists a need to provide a novel method, apparatus, and system for predicting a pulmonary arterial hypertension condition of a subject that can address the above issues.

Furthermore, other desirable features and characteristics will become apparent from the subsequent detailed description and the appended claims, taken in conjunction with the accompanying drawings and this background of the disclosure.

### SUMMARY

In a first aspect, the present disclosure provides a method for predicting a pulmonary arterial hypertension condition of a subject, the method comprising: measuring, by a processor, a morphological feature of a region of a heart of the subject from a plurality of echocardiogram videos, each of the plurality of echocardiogram videos showing the heart at a different viewing angle, the morphological feature relating to the pulmonary arterial hypertension condition; and generating, by the processor, a prediction score representing a likelihood of the subject having the pulmonary arterial hypertension condition from the morphological feature.

In a second aspect, the present disclosure provides an apparatus for predicting a pulmonary arterial hypertension condition of a subject, the apparatus comprising: at least one processor; and at least one memory including computer program code, the at least one memory and the computer program code configured to, with the at least one processor, cause the apparatus at least to: measure a morphological feature of a region of a heart of the subject from a plurality of echocardiogram videos, each of the plurality of echocardiogram videos showing the heart at a different viewing angle, the morphological feature relating to the pulmonary arterial hypertension condition; and generate a prediction score representing a likelihood of the subject having the pulmonary arterial hypertension condition from the morphological feature.

In a third aspect, the present disclosure provides a system for predicting a pulmonary arterial hypertension condition of a subject, the system comprising the apparatus according to the second aspect and an imaging device for capturing the plurality of echocardiogram videos.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

Embodiments and implementations are provided by way of example only, and will be better understood and readily apparent to one of ordinary skill in the art from the following written description, read in conjunction with the drawings, in which:

FIG. 1 shows a block diagram illustrating a system for predicting a pulmonary arterial hypertension condition of a subject according to various embodiments of the present disclosure.

FIG. 2 shows a block diagram illustrating various components of an apparatus for predicting a pulmonary arterial hypertension condition of a subject according to an embodiment of the present disclosure.

FIG. 3 shows a flow chart illustrating a method for predicting a pulmonary arterial hypertension condition of a subject according to an embodiment of the present disclosure.

FIG. 4 shows a schematic diagram illustrating a model for predicting a pulmonary arterial hypertension condition of a subject according to various embodiments of the present disclosure.

FIG. 5 shows a flow chart illustrating an inference process according to an embodiment of the present disclosure.

FIG. 6 shows a flow chart illustrating a knowledge distillation process of according to an embodiment of the present disclosure.

FIG. 7 shows a flow chart illustrating student model training and teacher model training processes according to an embodiment of the present disclosure.

FIG. 8 shows a schematic diagram of an exemplary computing device suitable for use to execute the method of FIG. 3 and implement the apparatus of FIG. 1.

FIG. 9 shows exemplary images of multi-view echocardiogram videos showing different regions of the hearts of five different patients at four different viewing angles according to an embodiment of the present disclosure.

FIG. 10 shows exemplary images of multi-view echocardiogram videos showing the hearts of three patients with and without model balancing and results of features extracted from the videos according to an embodiment of the present.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of this invention to provide a method, apparatus, and system for predicting a pulmonary arterial hypertension (PAH) condition of a subject.

Where reference is made in any one or more of the accompanying drawings to steps and/or features, which have the same reference numerals, those steps and/or features have for the purposes of this description the same function(s) or operation(s), unless the contrary intention appears.

It is to be noted that the discussions contained in the “Background” section and that above relating to prior art arrangements relate to discussions of methods which form public knowledge through their use. Such methods should not be interpreted as a representation by the present inventor(s) or the patent applicant that such methods in any way form part of the common general knowledge in the art.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilising terms such as “receiving”, “calculating”, “determining”, “updating”, “generating”, “initialising”, “outputting”, “receiving”, “retrieving”, “identifying”, “dispersing”, “authenticating” or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialised apparatus to perform the required method steps may be appropriate. The structure of a computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM mobile telephone system. The computer program when loaded and executed on such a computer effectively results in an apparatus that implements the steps of the preferred method.

Various embodiments of the present disclosure relate to a method, an apparatus and a system for predicting a medical condition of a subject, and more particularly a PAH condition of a subject.

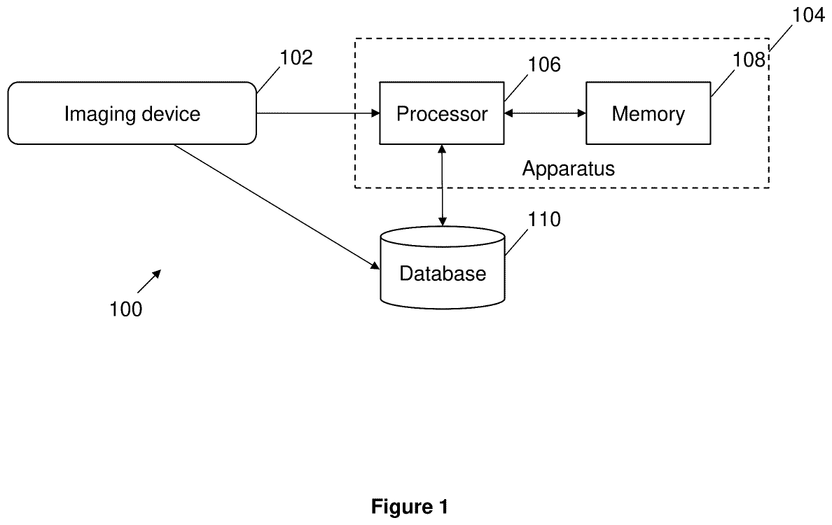

FIG. 1 shows a block diagram illustrating an apparatus 104 for predicting a PAH condition of a subject according to various embodiments of the present disclosure.

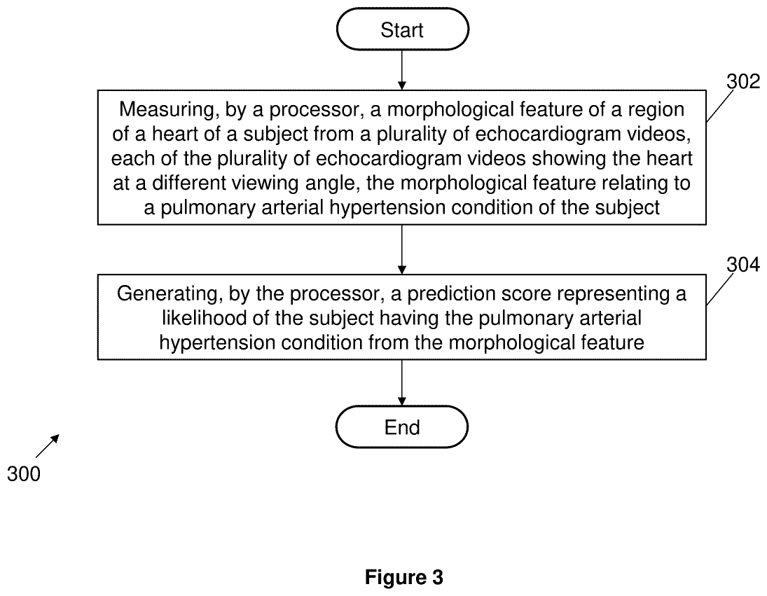

The managing of image or video input is performed by at least one imaging device 102 and an apparatus 104. For the sake of simplicity, only one imaging device 102 is illustrated. The system 100 comprises an imaging device 102 in communication with the apparatus 104. In an implementation, the apparatus 104 may be generally described as a physical device comprising at least one processor 106 and at least one memory 108 including computer program code. The at least one memory 108 and the computer program code are configured to, with the at least one processor 106, cause the physical device to perform the operations described in FIG. 3. The processor 106 is configured to receive one or more images or videos from the imaging device 102 or retrieve one or more images or videos from a database. Alternatively or additionally, the one or more images or videos captured by the imaging device 102 is stored in a database 110, and the processor 106 is configured to retrieve the one or more images or videos from the database 110. It should be appreciated that the imaging device 102 may be a part of the apparatus 104, forming a system 100 to perform operations described in FIG. 3.

The imaging device 102 may be an ultrasound imaging device such as an echocardiograph which provides a variety of data such as echocardiogram data relating to a region of a heart of a subject. In an implementation, data derived from the imaging device 102 may be stored in memory 108 of the apparatus 104 or a database 110 accessible by the apparatus 104. The data may include (i) data relating to a morphological feature of a region of a heart, such as diameter, area, volume, and/or wall thickness of left ventricle, right ventricle, left atrium, right atrium, inferior vena cava, aorta, pulmonary artery, mitral valve, pulmonary valve, or a combination thereof, and (ii) data relating to a velocity of blood flow in the region of the heart such as tricuspid regurgitation (TR) velocity, pulmonary regurgitation (PR) velocity, or a combination thereof.

In an implementation, imaging data such as subject identifier, location, and resolution, and/or time data which includes a timestamp at which the region of the heart is identified may also be derived from the imaging device 102. The imaging data and/or time data may be stored in memory 108 of the apparatus 104 or a database 110 accessible by the apparatus 104 and the processor 106 is configured to identify and retrieve data, image, or video based on the time data. It should be appreciated that the database 110 may be a part of the apparatus 104.

The apparatus 104 may be configured to communicate with the imaging device 102 and the database 110. In an example, the apparatus 104 may receive, from the imaging device 102, or retrieve from the database 110, one or more echocardiogram videos showing a heart of a subject at a viewing angle. For example, the one or more echocardiogram videos may show at least one of: a parasternal left ventricle long axis view, a pulmonary artery long axis view, a left ventricular short axis view, and an apical four-chamber heart view.

As mentioned above, early detection of PAH is crucial. However, existing PAH diagnostic methods may not be suitable for routine screening purposes due to their reliance on highly skilled physicians, variability of interpretation, radiation exposure, long scanning time required, and/or potential inaccuracies.

The various embodiments described in the present disclosure provides a non-invasive and cost-effective method, apparatus, and system that utilises multi-view echocardiogram videos to predict a PAH condition of a subject. This approach offers improved PAH prediction that is accurate, reliable, and suitable for use in routine screening of PAH.

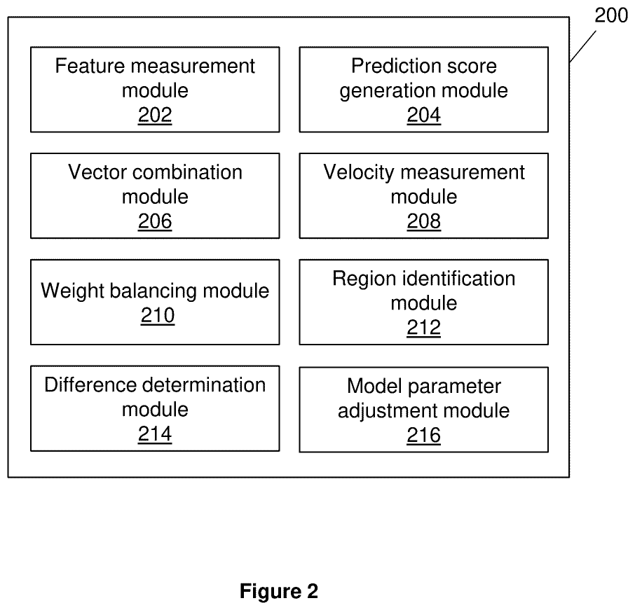

FIG. 2 shows a block diagram illustrating various components of an apparatus 200 for predicting a PAH condition of a subject according to an embodiment of the present disclosure. The apparatus 200 may comprise a feature measurement module 202, a prediction score generation module 204, a vector combination module 206, a velocity measurement module 208, a weight balancing module 210, a region identification module 212, a difference determination module 214 and a model parameter adjustment module 216. In an alternative embodiment, such components or modules are comprised on, or implemented as part of, a processor of the apparatus 200.

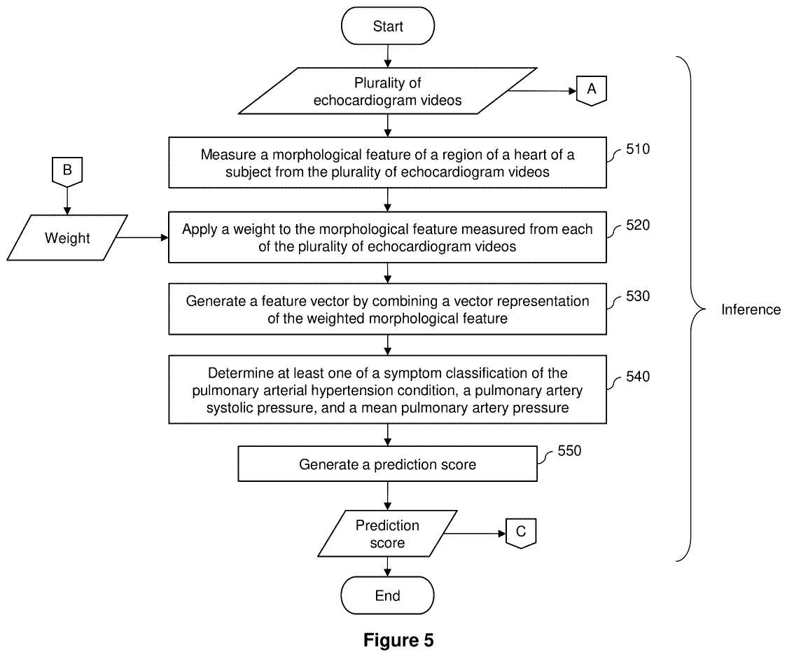

As shown in the exemplified method for predicting a PAH condition of a subject in FIG. 3 and the flow charts in FIGS. 5 to 7, the apparatus 200 or a processor of the apparatus 200, when in operation, is configured to perform the following steps:
step 302: measuring a morphological feature of a region of a heart of the subject from a plurality of echocardiogram videos, each of the plurality of echocardiogram videos showing the heart at a different viewing angle, the morphological feature relating to the pulmonary arterial hypertension condition (e.g., step 510 in FIG. 5);
step 304: generating a prediction score representing a likelihood of the subject having the pulmonary arterial hypertension condition from the morphological feature (e.g., step 550 in FIG. 5); and

The feature measurement module 202 is configured to perform step 302, while the prediction score generation module 204 is configured to perform step 304.

Additionally or alternatively, the vector combination module 206 may be configured to generate a feature vector by combining a vector representation of the morphological feature measured from each of the plurality of echocardiogram videos. The prediction score generation module 204 may then be configured to generate the prediction score from the feature vector (e.g., step 550 in FIG. 5).

Alternatively and additionally, the velocity measurement module 208 may be configured to measure a velocity of blood flow in the region of the heart from the plurality of echocardiogram videos, and the prediction score generation module 204 may be configured to generate the prediction score from the morphological feature and the velocity of blood flow.

Alternatively and additionally, the prediction score generation module 204 may be configured to: determine at least one of symptom classification of the pulmonary arterial hypertension condition, a pulmonary artery systolic pressure, and a mean pulmonary artery pressure from the morphological feature (e.g., step 540 in FIG. 5); and generate the prediction score from the at least one of the symptom classification of the pulmonary arterial hypertension condition, the pulmonary artery systolic pressure, and the mean pulmonary artery pressure (e.g., step 550 in FIG. 5). For example, symptoms of pulmonary arterial hypertension may be classified into four different types, based on severity, namely: (i) non-PAH (mPAP≤20 mmHg), (ii) mild (20 mmHg≤mPAP<35 mmHg), (iii) intermediate (35 mmHg≤mPAP<50 mmHg), and (iv) severe (50 mmHg≤mPAP).

Additionally or alternatively, the weight balancing module 210 may be configured to apply a weight to the morphological feature measured from each of the plurality of echocardiogram videos (e.g., step 520 in FIG. 5); and the vector combination module 206 may then be configured to generate the feature vector by combining a vector representation of the weighted morphological feature from each of the plurality of echocardiogram videos (e.g., step 530 in FIG. 5).

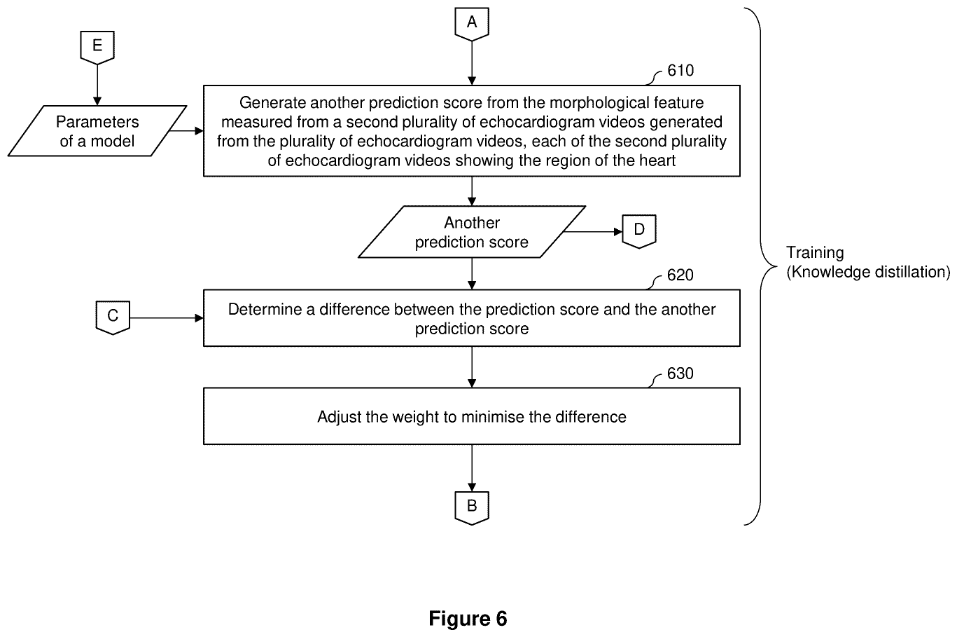

Additionally or alternatively, the prediction score generation module 204 may be configured to: generate another prediction score from the morphological feature measured from a second plurality of echocardiogram videos, the second plurality of echocardiogram videos generated from the plurality of echocardiogram videos to identify the region of the heart, each of the second plurality of echocardiogram videos showing the region of the heart (e.g., step 610 in FIG. 6). Then, the difference determination module 214 may be configured to determine a difference between the prediction score and the another prediction score (e.g., step 620 in FIG. 6), and the model parameter adjustment module 216 may be configured to adjust the weight to minimise the difference (e.g., step 630 in FIG. 6).

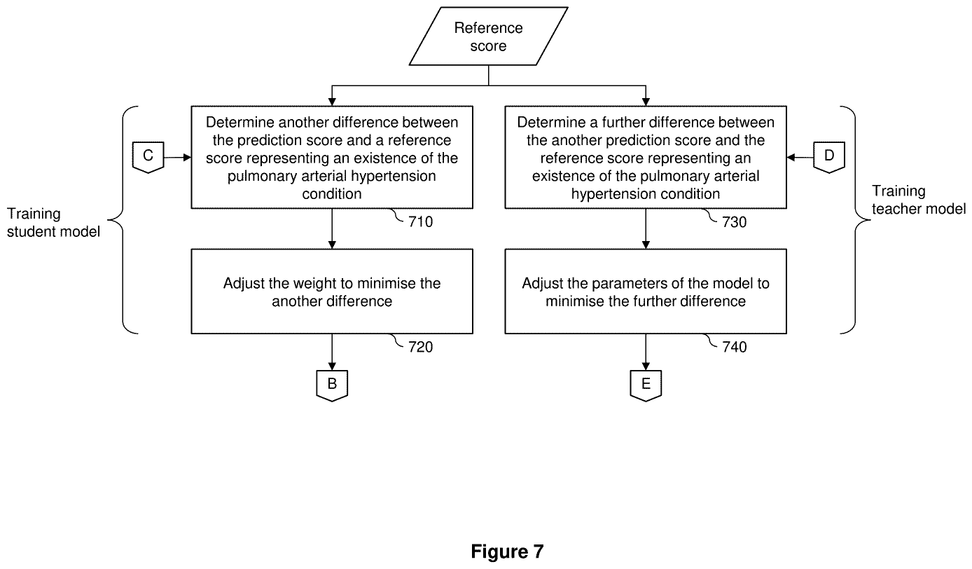

Additionally or alternatively, the difference determination module 214 may be configured to determine another difference between the prediction score and a reference score representing an existence of the pulmonary arterial hypertension condition (e.g., step 710 in FIG. 7), and the model parameter adjustment module 216 may be configured to adjust the weight to minimise the another difference (e.g., step 720 in FIG. 7).

Additionally, or alternatively, the prediction score generation module 204 may be configured to generate the another prediction score using parameters of a model in step 610. Then the difference determination module 214 may be configured to determine a further difference between the another prediction score and the reference score (e.g., step 730 in FIG. 7), and the parameter adjustment module 216 may be configured to adjust the parameters of the model to minimise the further difference (e.g., step 740 in FIG. 7).

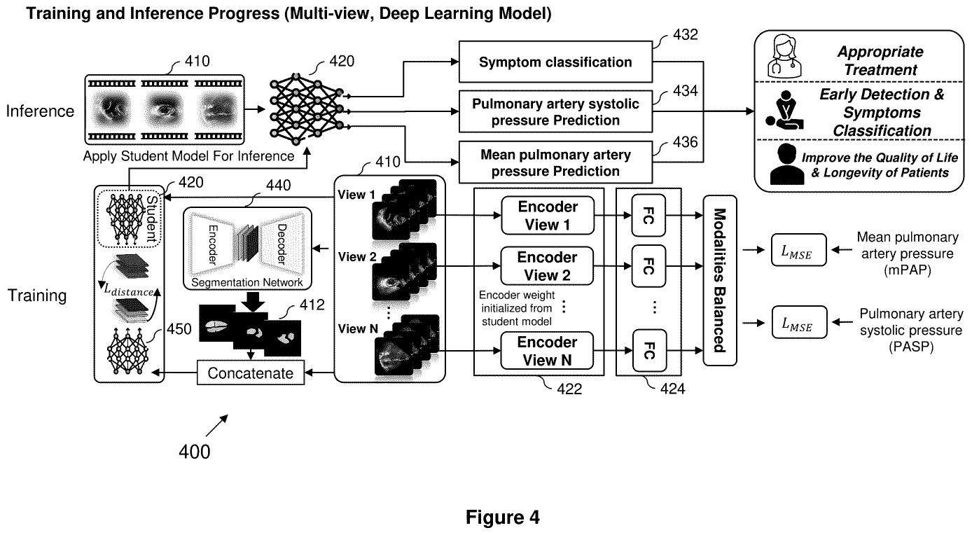

FIG. 4 shows a model 400 for predicting a pulmonary arterial hypertension condition of a subject using a plurality of echocardiogram videos according to an embodiment of the present disclosure. For example, the model 400 may automatically predict the mean pulmonary artery pressure (mPAP) of patients and classify the symptoms (e.g., non-PAH, mild, intermediate, or severe) from multi-view echocardiography videos. As mentioned above, single-view echocardiogram videos may provide an incomplete representation of cardiac morphology, as different views (e.g., parasternal long-axis view, parasternal short-axis view, and apical four-chamber view) highlight different aspects of the anatomy of the heart. Consequently, different views may provide varying visual representations of the same heart. For example, one view might provide a clear image of the left ventricle and its function, while another view might better show the atria and the valves. To achieve a comprehensive and accurate evaluation, it is crucial to integrate echocardiogram videos from multiple views. This multi-view approach, as shown in FIG. 4, allows for a more thorough analysis by providing a holistic understanding of the heart's morphology and function, helping to identify conditions that might be missed or mischaracterised when using only single-view echocardiogram videos. The operations of the model 400 can be categorised into two main processes-a training process and an inference process.

As shown in FIG. 4, the model 400 comprises various training stages for knowledge distillation, which transfers knowledge from a larger teacher model to a smaller student model. In particular, the teacher model is first trained and then the student model is trained using the teacher model. A multi-view neural network (e.g., convolutional neural network 420) is deployed to fuse information from multiple views of cardiac (e.g., combining a vector representation of the morphological feature measured from each of the plurality of echocardiogram videos) and is able to generate low-dimensional and high-dimensional features from raw information. For example, the high-dimensional features may refer to semantic Information including the morphological feature, and the low-dimensional features may include details such as the edge and texture information. The encoder structure (e.g., ResNet2D+1D) remains the same and parameters are trained independently for each view. Furthermore, in order to integrate features from different views, the modalities balanced method is employed to balance weights and contributions across multi-view (e.g., applying a weight to the morphological feature measured from each of the plurality of echocardiogram videos). Projection layers may then be used for both mPAP prediction and PAH classification (e.g., determining at least one of a symptom classification of the pulmonary arterial hypertension condition, a pulmonary artery systolic pressure, and a mean pulmonary artery pressure from the morphological feature). The projection layers may be layers (e.g., fully connected layers) in the neural network that are used to reduce the dimension of output features. For example, the network may generate a fixed number of features (e.g., 64, 128, 256 or 512). If the task requires classifying the PAH condition into 4 different classes, the projection layers may reduce this number of features to 4. The channel with the highest score among the 4 may then be selected as the final output.

Knowledge distillation is introduced to distil morphological information from cardiac according to the ventricular and atrial segmentation (e.g., by generating another prediction score from the morphological feature measured from a second plurality of echocardiogram videos generated from the plurality of echocardiogram videos to identify the region of the heart, each of the second plurality of echocardiogram videos showing the region of the heart; determining a difference between the prediction score and the another prediction score; and adjusting the weight to minimise the difference) and further lighten the computational consumption and memory cost of training and inference. In this module, feature maps from teacher models (e.g., another prediction score) are used as the distillation target for student models and a loss (e.g., difference between the prediction score and the another prediction score) is introduced to narrow representation (e.g., adjusting the weight to minimise the difference) that carries morphology information of key structures of the heart such as atria and ventricles.

### Inference

The following describes implementations of the inference process according to steps 510 to 550 as shown in FIG. 5.

As an example, steps 510 to 550 in FIG. 5 relating to the inference process may be implemented by a convolutional neural network (CNN) 420 comprised by the model 400.

The CNN 420 (e.g., ResNet2D+1D) takes in a plurality of echocardiogram videos 410 (e.g., multi-view echocardiogram videos) showing the heart of a subject as input and generates a prediction score representing a likelihood of the subject having a PAH condition. There are N number of echocardiogram videos 410, each showing the heart from a different view (e.g., View 1, View 2, . . . , View N). The CNN 420 comprises N number of encoders 422 for independent processing of each of the echocardiogram videos 410 to measure a morphological feature of a region of the heart from each video (step 510). During processing, each encoder 422 may apply a different weight to each morphological feature measured from the plurality of echocardiogram videos 410 (step 520). The morphological features are represented as vectors, which are then concatenated into a feature vector (step 530) by passing through a fully connected layer 424.

In some examples, the CNN 420 may determine, based on the feature vector, at least one of a symptom classification of the pulmonary arterial hypertension condition 432, a pulmonary artery systolic pressure 434, and a mean pulmonary artery pressure 436 (step 540), and then generate the prediction score (step 550).

In some embodiments, the CNN 420 may also measure a velocity of blood flow in the region of the heart (e.g., tricuspid regurgitation (TR) velocity, pulmonary regurgitation (PR) velocity) from the plurality of echocardiogram videos 410 and generate the prediction score from the morphological features and the velocity of blood flow measured from the echocardiogram videos 410.

It is hypothesised that complementary information exists among different views, and that the accuracy for symptom classification and mPAP prediction can be boosted by fusing their features from these views. Each view presents unique characteristics of the structure and function of the heart, and individual hearts may have subtle differences in shape, size, and response to physiological conditions due to factors such as genetics, lifestyle, and health status. Understanding these individual variations is crucial for accurate diagnosis and treatment in cardiology. For patients with PAH, the structure and function of their heart can differ significantly compared to those without the condition, as PAH is characterised by high blood pressure in the pulmonary arteries, which can lead to significant changes in the heart's structure and function. Various embodiments of the present disclosure uses multi-view echocardiogram videos which provide a more complete representation of the heart from different viewing angles, and integrates features from various views, thereby complementing the deficiency of information and allowing for more accurate measurements of morphological features and blood flow velocities. Accordingly, this reduces the potential inaccuracies associated with existing methods that use single-view echocardiogram videos and improves the overall reliability and accuracy of the PAH prediction.

### Training

The following paragraphs explain the training process of the model 400, and are implementations of the knowledge distillation according to steps 610 to 630 as shown in FIG. 6 and model training processes according to steps 710 to 740 as shown in FIG. 7.

As an example, the model 400 utilises knowledge distillation to accelerate the inference speed and enhance the performance of PAH prediction and reduce its computational consumption. To do so, the model 400 comprises a teacher model 450 to transfer morphological information to the student model, CNN 420, during the training process. The teacher model 450 receives a combination of the plurality of echocardiogram videos 410 (e.g., RGB or greyscale echocardiogram videos) and a plurality of segmented echocardiogram videos 412 showing different identified regions of the heart (e.g., organ-based segmentation flow). Meanwhile, the student model 420 only receives the plurality of echocardiogram videos 410. The size of the video inputted into each model may be denoted by H×W×T×C, where H and W represents the height and width of each video frame, C represents the number of input channels of the videos (e.g., RGB echocardiogram video contains three input channels-red, green, and blue; while greyscale echocardiogram video contains only one input channel), and T represents the temporal length, which is indicated by the number of video frames. As an example, the size (H×W×T×C) of each echocardiogram video inputted into the teacher model 450 and the student model 420 may be 112×112×32×2 (e.g., a concatenation of greyscale echocardiogram video and segmented echocardiogram video) and 112×112×32×1 (e.g., a greyscale echocardiogram video), respectively.

In one implementation, the teacher model 450 processes the echocardiogram videos 410 and segmented echocardiogram videos 412 to generate another prediction score (step 610). The output of the teacher model is compared against the output of the student model 420 (step 620) to determine a difference, and parameters of the student model 420 (e.g., weight) are adjusted during the training process to minimise the difference (step 630).

The model 400 may also comprise a segmentation model 440 to generate the organ-based segmentation flow according to the echocardiogram videos 410, which is in turn used to train the teacher model 440. The segmentation model 440 takes in the plurality of echocardiogram videos 410 as input, identifies different regions of the heart, and generates segmented echocardiogram videos 412 showing the identified regions of the heart. For example, the backbone of the segmentation model 440 may be ResNet, and its input size may be 112×112×32×1.

For example, the multi-view model may comprise N models for N different views. Each model corresponds to a different view and uses the same type of network as the student model (e.g., ResNet2D+1D) with the same video input size of 112×112×32×1 to generate a feature representation of the input video. The outputs from each model are then concatenated into a feature vector (e.g., a vector of N×Feature size) through a fully connected layer, which is used to predict PAH.

In some implementations, both the teacher model 440 and student model 420 are further trained using the same training configuration (steps 710 to 740). For example, stochastic gradient descent (SGD) and cosine annealing scheduler may be used as the optimiser in training. The learning rate may be set to 0.001 and the training may be completed in 70 epochs.

As an example, each of the prediction score generated by the student model 420 and the teacher model 450 is compared against a reference score (e.g., a ground truth label representing an existence of the PAH condition) to determine a difference (e.g., a distance between the prediction score and the ground truth label) (steps 710, 730), and the parameters (e.g., weight) of the student model 420 and the teacher model 450 are adjusted during the training process to minimise the difference (steps 720, 740).

The distances between the prediction scores and ground truth labels representing an existence of the PAH condition are measured using mean squared error (MSE), which serves as the loss function (steps 620, 710, 730).

For fair evaluation, data may be split into different subsets for used in training, validation, and testing phases in the ratio of 8:1:1. During the training phase, the machine learning model learns to recognise patterns in the data by adjusting its parameters to minimise the error between its predictions and the actual values. A large portion of the available data, known as the training dataset, is used in this phase. The model iteratively processes this training dataset, adjusting weights and biases through algorithms such as backpropagation in neural networks. The validation phase is used to tune the model's hyperparameters and prevent overfitting. A separate subset of the data, known as the validation dataset, is used in this phase to assess how well the model generalises to unseen data. The model's performance on this validation dataset is monitored during training, and hyperparameters are adjusted accordingly. Techniques like cross-validation may be employed to ensure robustness. The testing phase evaluates the model's performance on completely unseen data to estimate its real-world performance. The testing dataset, which has not been used in either training or validation, is used in this phase to provide an unbiased evaluation of the model's final performance. Some examples of reported metrics may include accuracy, precision, recall, F1 score, and area under the curve of the receiver operating characteristic curve (AUC-ROC). An external dataset from a different data centre may be used for the external dataset testing to validate the model's robustness and generalisability on data from different sources or populations. Finally, the results inferred from the testing dataset by the model with the best performance during the validation phase in 60-70 epochs are reported. This final step consolidates the model's performance metrics (e.g., summarising its performance across all datasets and document findings) and prepares for deployment or publication. In particular, the reporting of the result inference may provide insights and conclusions based on the model's performance.

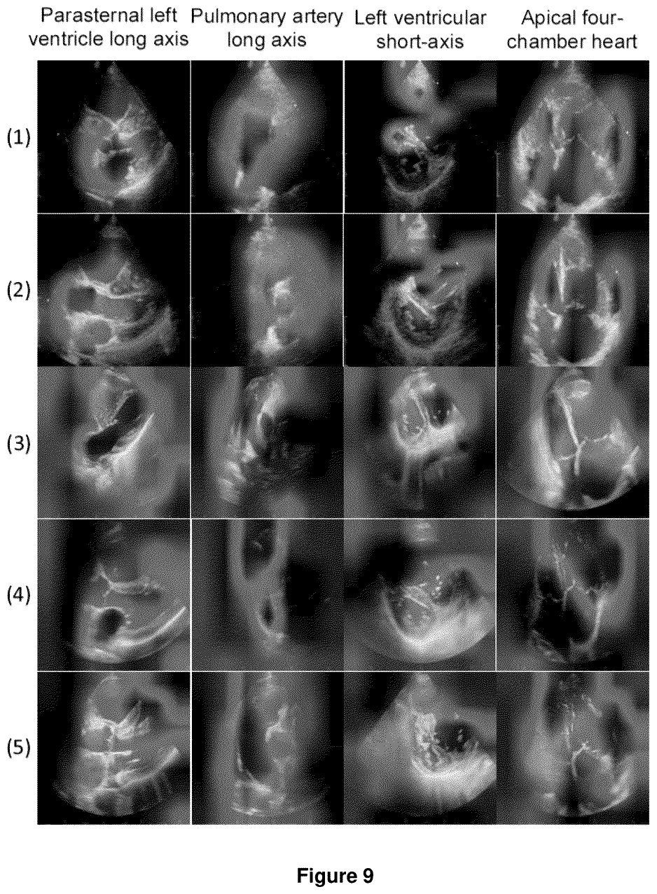

The present disclosure introduces a multi-views model, which comprises feature extractors (e.g., N models) for different views with the same structure (e.g., ResNet 2D+1D), as well as a fully connected layer for predicting the classification of the PAH symptoms and mPAP, where all the feature extractors of different views are trained synchronously. During the training process, the model assumes that the content of all views is equally important for PAH prediction, and contribution of each view to the overall multi-views model is considered the same. However, each feature extractor of each view can also individually predict the PAH and the mPAP, and different views may present different results in PAH prediction. For example, referring to FIG. 9, the apical four chamber heart view is able to achieve the best result among all views. For the coverage speed, the apical four-chamber heart view takes the shortest training time to converge to the optimal value, indicating that certain views are more accessible for deep learning models to learn from. For example, the apical four chamber heart view may be easier to converge to the optimal value as compared to other views. As a result, when these views are used as input for the multi-view model, they act as a ‘shortcut’ for network training, leading to faster coverage during training (e.g., the model requires less time to coverage to the considerable results) but potentially lower accuracy during testing. This phenomenon highlights the challenge of unbalanced training, where different views provide different information that may be difficult to be balanced. While all view were assumed to contribute equally to the model, in reality, some views may contribute more significantly, potentially causing the model to rely too heavily on those views and overlook valuable information from other views.

Existing methods attempting to solve the challenge from the unbalanced training are limited to balancing modality learning between only two different modalities (e.g., audio and video). In contrast, the present invention provides an improved method that expands this capability to balance learning across N modalities.

As an example, model balancing method may be used to further improve the ability of the model to extract features from multi-view echocardiogram videos, by balancing the weights and contribution of the different modalities to output of the model during the training process (e.g., applying a weight to the morphological feature measured from each of the plurality of echocardiogram videos). This prevents the model 400 from taking “shortcuts” during training, thereby improving the accuracy of the PAH prediction by the model.

In one implementation, the model 400 is configured to process four modalities, e.g., four echocardiogram videos 410 showing the heart at four different viewing angles for PAH prediction. Each echocardiogram video may have a spatial resolution of 112×112 and temporal length of 48 frames. FIG. 9 shows exemplary images of multi-view echocardiogram videos 410 showing different regions of the hearts of five different patients at four different viewing angles: a parasternal left ventricle long axis view, a pulmonary artery long axis view, a left ventricular short axis view, and an apical four-chamber heart view.

The model 400 is trained using a plurality of echocardiogram videos. Each training dataset comprises a plurality of echocardiogram videos and is denoted by X={x₁, x₂, . . . , xₙ}, where n indicates the number of modalities (e.g., views) and xₙ indicates an echocardiogram video of the n-th modality (e.g., n-th view), x∈ [symbol] . Encoder E={φ¹, φ², . . . , φⁿ} comprises a plurality of independent encoders φ for feature extraction without sharing any parameters. Here, φⁿ indicates the encoder that accepts input from the corresponding echocardiogram video xₙ.

A late fusion method is used to concatenate features extracted by encoder E via a fully connected layer denoted by W∈ [symbol] ^{×(x}^{₁}^{+x}^{₂}^{+ . . . +x}^{ₙ}⁾ with a bias denoted by b∈ [symbol] , where P indicates the number of categories (e.g., number of symptoms of PAH) or predicted scores (e.g., mPAP). The logits output (e.g., the prediction score) of the model 400 is denoted by [symbol] (x):

[symbol]

(x)=W[φ¹(x₁),φ²(x₂), . . . ,φⁿ(xₙ)]+b (1)

Equation (1) can be rewritten to represent the combination of the contribution to the logits output from each modality:

[symbol]

(x)=W¹·φ¹(x₁)+W²·φ²(x₂)+ . . . +Wⁿ·φⁿ(xₙ)+b (2)

The sub logits output, which represents the individual contribution of the n-th modality to the logits output, is denoted by f(xₙ):

$$f ( {x}_{n} ) = {W}^{n} \cdot {φ}^{n} + \frac{b}{n} \qquad ( 3 )$$

MSE is used as the loss function the training process. The MSE of the logits output [symbol] (x) is denoted by:

$${L}_{All} = \frac{1}{M} {\sum }_{j = 1}^{M} {( {g}_{j} - f ( {x}_{j} ) )}^{2} ,$$

while the MSE of each sub logits output [symbol] (xₙ) is denoted by:

$${L}_{n} = \frac{1}{M} {\sum }_{j = 1}^{M} {( {g}_{j} - f ( {x}_{nj} ) )}^{2} ,$$

where g denotes the ground truth label representing an existence of the PAH condition and the M denotes the sample quantity.

The MSE indicates the gap between the prediction scores and ground truth labels. A higher MSE indicates that a modality is more difficult to be trained, and the model 400 is less able to accurately predict the PAH condition from that modality. Conversely, a lower MSE indicates that a modality is more efficiently trained, leading to faster convergence.

In some embodiments, a model balancing method is used to tune the training process in order to counter the tendency of the optimiser (e.g., SGD) to train modalities that are easier to converge. The weight of the fully connected layer Wⁿ and the parameters of encoder φⁿ are updated according to the below equation:

$${W}_{t + 1} = {W}_{t} - η \frac{\partial {L}_{All}}{\partial f ( x )} = {W}_{t} - η \frac{2}{n} {\sum }_{i = 1}^{M} ( {W}_{j}^{n} \cdot {φ}_{j}^{n} - {g}_{j}^{n} ) \qquad ( 4 )$$

In equation (4), η denotes the learning rate. For each modality, equation (4) can be rewritten as:

$${W}_{t + 1}^{n} = {W}_{t}^{n} - η 2 ( {W}_{j}^{n} \cdot {φ}_{j}^{n} - {g}_{j}^{n} ) ,$$

indicating that the optimisation of the model for each modality has no correlation with the optimisation of the model for other modalities.

The MSE for each modality can be written as

$${L}_{n} = \frac{1}{M} {\sum }_{j = 1}^{M} {( {g}_{j} - f ( {x}_{nj} ) )}^{2} ,$$

and treated as the discrepancy of modality contribution to the learning object (e.g., the logits output or the prediction score). As mentioned above, since each view provides different information, the network may extract information from each view in the fastest convergence path. Inevitably, some views may provide richer and more direct information, while other views may present different information characteristics and are more difficult to extract. Therefore, in the present implementation, the sub logits output (e.g., contribution of each view to the classification of PAH symptoms) is calculated, and the model is balanced to ensure that the network can pay more attention to those views that are more difficult to extract information.

The model balancing method uses a discrepancy metric denoted by: R=Δdiag (V⁻¹V^{T}), where matrix V represents the mean squared error of each modality:

$$V = ( \begin{matrix}{L}_{1} \\ {L}_{2} \\ ⋮ \\ {L}_{n}\end{matrix} ) = \sqrt{\frac{1}{M} {\sum }_{j = 1}^{M} ( \begin{matrix}{( {W}_{j}^{1} \cdot {φ}_{j}^{1} - {g}_{j}^{1} )}^{2} \\ {( {W}_{j}^{2} \cdot {φ}_{j}^{2} - {g}_{j}^{2} )}^{2} \\ ⋮ \\ {( {W}_{j}^{n} \cdot {φ}_{j}^{n} - {g}_{j}^{n} )}^{2}\end{matrix} )} \qquad ( 5 )$$

The contribution discrepancy is measured by taking the first row of matrix

$$R = ( {ω}_{t}^{1} , {ω}_{t}^{2} , … , {ω}_{t}^{n} )$$

and dividing it by the min R, where ω denotes learnable parameters of the encoders. The values corresponding to each modality denote the weight that needs to be assigned during training optimisation. Hence, the gradient is adaptively modulated according to:

$${k}_{t}^{n} = \{ \begin{matrix}1 - tanh ( α \cdot {ω}_{t}^{n} ) , & {ω}_{t}^{n} > 1 , \\ 1 & others\end{matrix} \qquad ( 6 )$$

The gradient is a vector of partial derivatives of the loss function with respect to parameters (e.g., weights and biases) of the model, and measures how much the loss function changes with a small change in each parameter. Here

$${k}_{t}^{n}$$

is the coefficient that control the update of the SGD optimiser in iteration t and a is the hyper-parameter to control the degree of modulation of the SGD optimiser.

This approach balances the training process between different modalities and prevents the model 400 from taking “shortcuts” during training, thereby improving the accuracy of the PAH prediction by the model.

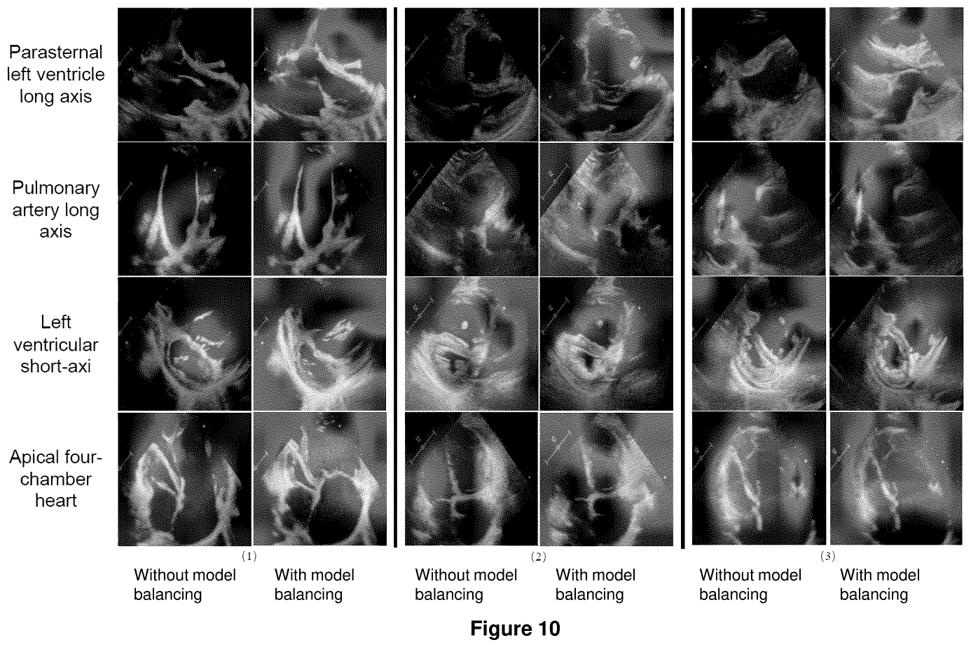

FIG. 10 shows exemplary images of multi-view echocardiogram videos showing the hearts of three patients with and without the model balancing and the results of features extracted from the videos according to an embodiment of the present disclosure. As can be seen from the results obtained without model balancing, echocardiogram videos showing the parasternal left ventricle long axis view and the pulmonary artery long axis view provide less contribution to the prediction of PAH, while echocardiogram videos showing the left ventricular short axis view, and apical four-chamber heart view dominate the result. Introduction of model balancing during the training enables the model to pay more attention to the parasternal left ventricle long axis view and the pulmonary artery long axis view, thereby improving feature extraction ability of the model.

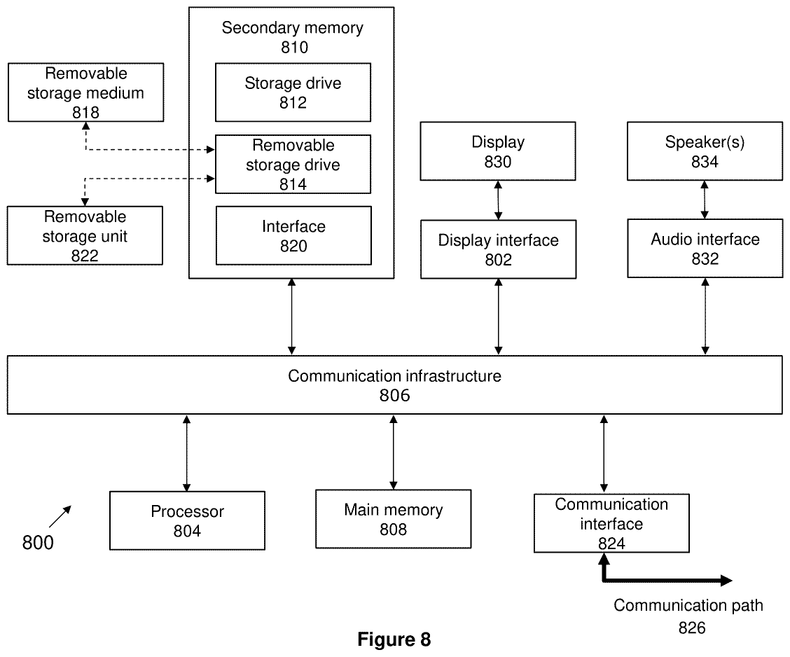

FIG. 8 depicts an exemplary computing device 800, hereinafter interchangeably referred to as a computer system 800, where one or more such computing devices 800 may be used to execute the method of FIG. 3. The exemplary computing device 800 can be used to implement the apparatus 104 shown in FIG. 1 or the apparatus 200 shown in FIG. 2. The following description of the computing device 800 is provided by way of example only and is not intended to be limiting.

As shown in FIG. 8, the example computing device 800 includes a processor 804 for executing software routines. Although a single processor is shown for the sake of clarity, the computing device 800 may also include a multi-processor system. The processor 804 is connected to a communication infrastructure 806 for communication with other components of the computing device 800. The communication infrastructure 806 may include, for example, a communications bus, cross-bar, or network.

The computing device 800 further includes a main memory 808, such as a random access memory (RAM), and a secondary memory 810. The secondary memory 810 may include, for example, a storage drive 812, which may be a hard disk drive, a solid state drive or a hybrid drive and/or a removable storage drive 814, which may include a magnetic tape drive, an optical disk drive, a solid state storage drive (such as a USB flash drive, a flash memory device, a solid state drive or a memory card), or the like. The removable storage drive 814 reads from and/or writes to a removable storage medium 818 in a well-known manner. The removable storage medium 818 may include magnetic tape, optical disk, non-volatile memory storage medium, or the like, which is read by and written to by removable storage drive 814. As will be appreciated by persons skilled in the relevant art(s), the removable storage medium 818 includes a computer readable storage medium having stored therein computer executable program code instructions and/or data.

In an alternative implementation, the secondary memory 810 may additionally or alternatively include other similar means for allowing computer programs or other instructions to be loaded into the computing device 800. Such means can include, for example, a removable storage unit 822 and an interface 820. Examples of a removable storage unit 822 and interface 820 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 822 and interfaces 820 which allow software and data to be transferred from the removable storage unit 822 to the computer system 800.

The computing device 800 also includes at least one communication interface 824. The communication interface 824 allows software and data to be transferred between computing device 800 and external devices via a communication path 826. In various embodiments of the inventions, the communication interface 824 permits data to be transferred between the computing device 800 and a data communication network, such as a public data or private data communication network. The communication interface 824 may be used to exchange data between different computing devices 800 which such computing devices 800 form part an interconnected computer network. Examples of a communication interface 824 can include a modem, a network interface (such as an Ethernet card), a communication port (such as a serial, parallel, printer, GPIB, IEEE 1394, RJ45, USB), an antenna with associated circuitry and the like. The communication interface 824 may be wired or may be wireless. Software and data transferred via the communication interface 824 are in the form of signals which can be electronic, electromagnetic, optical or other signals capable of being received by communication interface 824. These signals are provided to the communication interface via the communication path 826.

As shown in FIG. 8, the computing device 800 further includes a display interface 802 which performs operations for rendering images to an associated display 3130 and an audio interface 832 for performing operations for playing audio content via associated speaker(s) 834.

As used herein, the term “computer program product” may refer, in part, to removable storage medium 818, removable storage unit 822, a hard disk installed in storage drive 812, or a carrier wave carrying software over communication path 826 (wireless link or cable) to communication interface 824. Computer readable storage media refers to any non-transitory, non-volatile tangible storage medium that provides recorded instructions and/or data to the computing device 800 for execution and/or processing. Examples of such storage media include magnetic tape, CD-ROM, DVD, Blu-ray Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a PCMCIA card and the like, whether or not such devices are internal or external of the computing device 800. Examples of transitory or non-tangible computer readable transmission media that may also participate in the provision of software, application programs, instructions and/or data to the computing device 800 include radio or infra-red transmission channels as well as a network connection to another computer or networked device, and the Internet or Intranets including e-mail transmissions and information recorded on Websites and the like.

The computer programs (also called computer program code) are stored in main memory 808 and/or secondary memory 810. Computer programs can also be received via the communication interface 824. Such computer programs, when executed, enable the computing device 800 to perform one or more features of embodiments discussed herein. In various embodiments, the computer programs, when executed, enable the processor 804 to perform features of the above-described embodiments. Accordingly, such computer programs represent controllers of the computer system 800.

Software may be stored in a computer program product and loaded into the computing device 800 using the removable storage drive 814, the storage drive 812, or the interface 820. The computer program product may be a non-transitory computer readable medium. Alternatively, the computer program product may be downloaded to the computer system 800 over the communications path 826. The software, when executed by the processor 804, causes the computing device 800 to perform the necessary operations to execute the method as shown in FIG. 3.

It is to be understood that the embodiment of FIG. 8 is presented merely by way of example to explain the operation and structure of the apparatus 104 shown in FIG. 1 or the apparatus 200 shown in FIG. 2. Therefore, in some embodiments one or more features of the computing device 800 may be omitted. Also, in some embodiments, one or more features of the computing device 800 may be combined together. Additionally, in some embodiments, one or more features of the computing device 800 may be split into one or more component parts.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A method for predicting a pulmonary arterial hypertension condition of a subject, the method comprising:
measuring, by a processor, a morphological feature of a region of a heart of the subject from a plurality of echocardiogram videos, each of the plurality of echocardiogram videos showing the heart at a different viewing angle, the morphological feature relating to the pulmonary arterial hypertension condition; and
generating, by the processor, a prediction score representing a likelihood of the subject having the pulmonary arterial hypertension condition from the morphological feature.

2. The method of claim 1, further comprising:
generating, by the processor, a feature vector by combining a vector representation of the morphological feature measured from each of the plurality of echocardiogram videos; and
generating, by the processor, the prediction score from the feature vector.

3. The method of claim 1, further comprising:
measuring, by the processor, a velocity of blood flow in the region of the heart from the plurality of echocardiogram videos; and
generating, by the processor, the prediction score from the morphological feature and the velocity of blood flow.

4. The method of claim 1, further comprising:
determining, by the processor, at least one of a symptom classification of the pulmonary arterial hypertension condition, a pulmonary artery systolic pressure, and a mean pulmonary artery pressure from the morphological feature; and
generating, by the processor, the prediction score from the at least one of the symptom classification of the pulmonary arterial hypertension condition, the pulmonary artery systolic pressure, and the mean pulmonary artery pressure.

5. The method of claim 2, further comprising:
applying, by the processor, a weight to the morphological feature measured from each of the plurality of echocardiogram videos; and
generating, by the processor, the feature vector by combining a vector representation of the weighted morphological feature from each of the plurality of echocardiogram videos.

6. The method of claim 5, further comprising:
generating, by the processor, another prediction score from the morphological feature measured from a second plurality of echocardiogram videos, the second plurality of echocardiogram videos generated from the plurality of echocardiogram videos to identify the region of the heart, each of the second plurality of echocardiogram videos showing the region of the heart;
determining, by the processor, a difference between the prediction score and the another prediction score; and
adjusting, by the processor, the weight to minimise the difference.

7. The method of claim 6, further comprising:
determining, by the processor, another difference between the prediction score and a reference score representing an existence of the pulmonary arterial hypertension condition; and
adjusting, by the processor, the weight to minimise the another difference.

8. The method of claim 7, wherein the generating the another prediction score comprises generating the another prediction score using parameters of a model, the method further comprising:
determining, by the processor, a further difference between the another prediction score and the reference score; and
adjusting, by the processor, the parameters of the model to minimise the further difference.

9. The method of claim 1, wherein the plurality of echocardiogram videos comprises an echocardiogram video showing at least one of: a parasternal left ventricle long axis view, a pulmonary artery long axis view, a left ventricular short axis view, and an apical four-chamber heart view.

10. The method of claim 1, wherein the morphological feature comprises at least one of: a diameter of a ventricle, a diameter of a pulmonary artery, a diameter of an inferior vena cava, and an area of a right atrium.

11. An apparatus for predicting a pulmonary arterial hypertension condition of a subject, the apparatus comprising:
at least one processor; and
at least one memory including computer program code,
the at least one memory and the computer program code configured to, with the at least one processor, cause the apparatus at least to:
measure a morphological feature of a region of a heart of the subject from a plurality of echocardiogram videos, each of the plurality of echocardiogram videos showing the heart at a different viewing angle, the morphological feature relating to the pulmonary arterial hypertension condition; and
generate a prediction score representing a likelihood of the subject having the pulmonary arterial hypertension condition from the morphological feature.

12. The apparatus of claim 11, wherein the at least one memory and the computer program code is configured to, with the at least one processor, cause the apparatus at least to further:
generate a feature vector by combining a vector representation of the morphological feature measured from each of the plurality of echocardiogram videos; and
generate the prediction score from the feature vector.

13. The apparatus of claim 11, wherein the at least one memory and the computer program code is configured to, with the at least one processor, cause the apparatus at least to further:
measure a velocity of blood flow in the region of the heart from the plurality of echocardiogram videos; and
generate the prediction score from the morphological feature and the velocity of blood flow.

14. The apparatus of claim 11, wherein the at least one memory and the computer program code is configured to, with the at least one processor, cause the apparatus at least to further:
determine at least one of a symptom classification of the pulmonary arterial hypertension condition, a pulmonary artery systolic pressure, and a mean pulmonary artery pressure from the morphological feature; and
generate the prediction score from the at least one of the symptom classification of the pulmonary arterial hypertension condition, the pulmonary artery systolic pressure, and the mean pulmonary artery pressure.

15. The apparatus of claim 12, wherein the at least one memory and the computer program code is configured to, with the at least one processor, cause the apparatus at least to further:
apply a weight to the morphological feature measured from each of the plurality of echocardiogram videos; and
generate the feature vector by combining a vector representation of the weighted morphological feature from each of the plurality of echocardiogram videos.

16. The apparatus of claim 15, wherein the at least one memory and the computer program code is configured to, with the at least one processor, cause the apparatus at least to further:
generate another prediction score from the morphological feature measured from a second plurality of echocardiogram videos, the second plurality of echocardiogram videos generated from the plurality of echocardiogram videos to identify the region of the heart, each of the second plurality of echocardiogram videos showing the region of the heart;
determine a difference between the prediction score and the another prediction score; and
adjust the weight to minimise the difference.

17. The apparatus of claim 16, wherein the at least one memory and the computer program code is configured to, with the at least one processor, cause the apparatus at least to further:
determine another difference between the prediction score and a reference score representing an existence of the pulmonary arterial hypertension condition; and
adjust the weight to minimise the another difference.

18. The apparatus of claim 17, wherein the at least one memory and the computer program code is configured to, with the at least one processor, cause the apparatus at least to further:
generate the another prediction score using parameters of a model;
determine a further difference between the another prediction score and the reference score; and
adjust the parameters of the model to minimise the further difference.

19. The apparatus of claim 11, wherein the plurality of echocardiogram videos comprises an echocardiogram video showing at least one of: a parasternal left ventricle long axis view, a pulmonary artery long axis view, a left ventricular short axis view, and an apical four-chamber heart view.

20. The apparatus of claim 11, wherein the morphological feature comprises at least one of: a diameter of a ventricle, a diameter of a pulmonary artery, a diameter of an inferior vena cava, and an area of a right atrium.

21. A system for predicting a pulmonary arterial hypertension condition of a subject comprising an apparatus of claim 11 and an imaging device for capturing the plurality of echocardiogram videos.
